(19) United States
(12) Patent Application Publication
(10) Pub. No.: US 2022/0186280 A1
(43) Pub. Date: Jun. 16, 2022

(54) METHOD FOR TESTING BACTERIAL FILTRATION EFFICACY OF FABRICS

(71) Applicant: Sanko Tekstil Isletmeleri San. Ve Tic. A.S., Inegol - BURSA (TR)

(72) Inventors: Mehmet Erkan OZSEYHAN, Inegol - BURSA (TR); Halil AKBAS, Inegol - BURSA (TR); Fatih KONUKOGLU, Inegol - BURSA (TR); Mahmut OZDEMIR, Inegol - BURSA (TR); Esin KILICKAN, Inegol - BURSA (TR); Adnan KOCAMER, Inegol - BURSA (TR); Ahmet OZKAN, Inegol - BURSA (TR); Orhan YAYLA, Inegol - BURSA (TR)

(21) Appl. No.: 17/550,191

(22) Filed: Dec. 14, 2021

(30) Foreign Application Priority Data
Dec. 15, 2020 (EP) 20214273.3

(51) Int. Cl. C12Q 1/06; C12N 1/04; C12N 1/20

(52) U.S. Cl. CPC C12Q 1/06; C12N 1/20; C12N 1/04

(57) ABSTRACT

The present invention relates to method of testing the bacterial filtering efficiency of a fabric, the method including the steps of preparing a solution including bacteria, peptone water and NaCl, feeding said solution including bacteria to a nebulizer, generating an aerosol of said bacterial solution and flowing said solution through a cascade impactor to provide a plurality of bacteria colonies in a plurality of plates present in a plurality of stages of said cascade impactor, characterized in that the concentration of NaCl in the peptone water used to prepare said bacterial solution is in the range of 30 g/L to 150 g/L and in that the temperature of said cascade impactor is in the range of −15° C. to 15° C.

This non-provisional application claims priority to and the benefit of European Application No. 20214273.3 filed Dec. 15, 2020, the content of which is incorporated herein by reference in its entirety.

### Field of the Invention

The present invention relates to a method of testing fabrics, in particular, fabrics that are suitable for the production of respiratory face masks. More specifically, the present invention relates to method for testing the bacterial filtration efficacy (BFE) of fabrics.

### State of the Art

Personal respiratory masks, also known as “face masks”, “respiratory masks” or “filtering face masks”, are protective devices used to protect the wearer's respiratory system from airborne particles. Facial masks are in fact worn over the nose and mouth of the user to protect him from unwanted material suspended in the air. In some embodiments (namely those without a valve that let breath exit the mask) the mask also acts as a filter to prevent or reduce the leakage of any particles suspended in the user's breath to protect other people from possible infections of the person wearing mask. Known masks are typically made of non-woven fabric in two forms: a cup-shaped shape or a flat shape in which the non-woven fabric is partially folded on itself to be able to adapt to the shape of the face when worn. One type of flat mask is known as a “surgical mask”. Flat masks made of woven fabric are also known.

A mask requires the presence of straps or bands, preferably of elastic material, which generally are in the form of loops that pass around the user's ears or around the user's head to keep the mask in the desired position on the user's face. The straps or equivalent retaining means are typically made separately and are attached to the body of the mask, by means such as sewing, gluing, ultrasonic welding, stapling or other means commonly known to those skilled in the art. Protective devices are also known in which the retaining means are loops of elastic material attached to a folded portion of the mask body.

A face mask also typically requires the presence of a strip of plastically bendable material, generally a metal strip, that is located at the upper edge of the mask, i.e., at the side of the mask that is transversally floating over the bridge of the nose of the user when the mask is worn. This so-called “nose clip” may actually be made of any material provided it can be easily bended in a shape fitting to the bridge of the nose of the user to improve the air tightness of the mask.

In order to test the filtration efficacy of masks, the standard method according to EN 14683:2019+AC:2019 (EN 14683) is generally applied. According to EN 14683, bacterial filtration efficacy of masks is tested using a six-stage cascade impactor, and an aerosol chamber. A sample of the mask material is clamped between a six-stage cascade impactor and an aerosol chamber. An aerosol of Staphylococcus aureus (ATCC 6538) is produced by a nebulizer, introduced into the aerosol chamber, and drawn through the mask material and the impactor under vacuum. The bacterial filtration efficiency (BFE) of the mask is given by the number of colony forming units passing through the sample of mask material expressed as a percentage of the number of colony forming units present in the challenge aerosol. EN 14683 requires that the mean particle size (MPS) of the bacterial challenge, i.e., the mean particle size of the droplets containing the bacteria, when in contact with the cascade impactor, is maintained between 2.7 and 3.3 μm. The mean particle size of the droplets containing bacteria directly influences the data that are used to calculate the bacterial filtration efficiency of the mask. Therefore, a mean particle size that does not fall within the required range would provide a not reliable evaluation of the bacterial filtration efficiency of the mask.

Several problems are connected to EN 14683. One problem is that, whilst EN 14683 is used for testing masks made of woven fabrics, the same fabric may give different results from a test to another. Additionally, it is difficult to maintain the MPS of droplets containing bacteria in the required range when carrying out tests according EN 14683.A MPS lower than the required MPS (for example, in the range between 1.5 μm and 2.3 μm), will not satisfy the requirements of the standard EN 14683. Moreover, in view of the problem of maintaining the mean particle size of the droplets, also the formation of individual bacterial colonies on the petri dishes of the impactor and, as a consequence, the count of the colonies, results to be difficult in the MPS is not the required one.

### SUMMARY OF THE INVENTION

An aim of the present invention is to solve the above discussed problems and to provide a method for determining bacterial filtration efficiency of fabrics in a reliable way.

Another aim of the present invention is to provide a method for determining bacterial filtration efficiency of fabrics which allows to maintain the mean particle size of the droplets in the required range.

A further aim of the present invention is to provide a method for determining bacterial filtration efficiency of fabrics which is suitable to test woven fabrics, e.g., respiratory masks made of woven fabrics.

These and other aims are reached by the present invention that relates to a method for testing the bacterial filtration efficiency of a fabric, according to claim 1. The present invention also refers to a method for bringing into a required size range the Mean Particle Size of particles generated by a nebulizer in a device for testing Bacterial Filtration Efficiency of a fabric according to claim 7 and to a device for testing Bacterial Filtration Efficiency of a fabric according to claim 12. Preferred embodiments of the invention are object of the dependent claims.

In one embodiment, the present invention refers to a method of testing the bacterial filtering efficiency of a fabric, particularly in a cascade impactor, the method including the steps of preparing a solution including bacteria, peptone water and NaCl, feeding said solution including bacteria to a nebulizer, generating an aerosol of said bacterial solution and flowing said solution through said cascade impactor to provide a plurality of bacteria colonies in a plurality of plates present in a plurality of stages of said cascade impactor, characterized in that the concentration of NaCl in the peptone water used to prepare said bacterial solution is in the range of 30 g/L to 150 g/L and in that the temperature of said cascade impactor is in the range of −15° C. to 15° C.

In embodiments, the method is carried out according to EN 14683:2019+AC:2019 E modified to provide an NaCl concentration of the peptone water and a refrigeration of the cascade impactor as recited above.

In embodiments, the NaCl concentration is in the range of 60 g/L to 150 g/L, preferably in the range of 60 g/L to 120 g/L, more preferably of 100 g/L.

In embodiments, the temperature of the cascade impactor is in the range of −4° C. to 15° C., preferably in the range of 0° C. to 12° C.

In embodiments, in the testing conditions, the nebulizer is capable to generate an aerosol of a saline solution, free from bacteria, having Mean Particle Size in the range of 2.7 to 3.3 μm.

In embodiments, the nebulizer is selected from a jet nebulizer, an ultrasonic nebulizer, and a mesh nebulizer.

In an embodiment, the present invention further refers to a method for bringing into a required size range the Mean Particle Size of particles generated by a nebulizer in a device for testing Bacterial Filtration Efficiency of a fabric, the device comprising a nebulizer for generating an aerosol of a bacterial solution and a cascade impactor including a plurality of stages, said method comprising the steps of: generating an aerosol of a solution including bacteria, peptone water an NaCl; flowing said aerosol through the cascade impactor to provide a plurality of bacteria colonies in a plurality of plates present in the plurality of stages of said cascade impactor, characterized in comprising the steps of: performing a positive control run by feeding said aerosol to said cascade impactor; determining the Mean Particle Size of the aerosol particles based on the resulting number of bacterial colonies; comparing the obtained Mean Particle Size with a required range of Mean Particle Size; modifying the control run conditions by increasing or decreasing the concentration of NaCl in said bacterial solution and/or the temperature of said cascade impactor; performing at least another positive control run under said modified conditions and repeating said steps until the resulting Mean Particle Size is within the required range.

In embodiments, the mentioned required range of the Mean Particle Size is from 2.7 μm to 3.3 μm.

According to embodiments, the method for bringing into a required size range the Mean Particle Size of particles generated by a nebulizer in a device for testing Bacterial Filtration Efficiency of a fabric is carried out according to EN 14683:2019+AC:2019 E, optionally the run conditions being modified by increasing or decreasing the concentration of NaCl in said bacterial solution and/or the temperature of said cascade impactor.

In embodiments, bacteria are selected from the group consisting of Mycobacterium tuberculosis, Streptococcus pneumoniae, Legionella pneumophilla, Staphylococcus aureus, Bacillus subtilis, and Escherichia coli, preferably is Staphylococcus aureus, more preferably is Staphylococcus aureus ATCC 6538.

In embodiments, the fabric sample is placed between the first stage and the inlet cone of said cascade impactor.

In one embodiment, the present invention further refers to a device for testing Bacterial Filtration Efficiency of a fabric, the device comprising a nebulizer for generating an aerosol of a bacterial solution and a cascade impactor including a plurality of stages, characterized in comprising refrigerating means to refrigerate the cascade impactor.

In embodiments, the device is a device according to EN 14683:2019+AC:2019 E, preferably provided with refrigerating means to refrigerate the cascade impactor.

In embodiments, the refrigerating means are provided at the exit portion of said cascade impactor.

### DETAILED DISCLOSURE OF THE INVENTION

The present invention relates to a method of testing the bacterial filtering efficiency of a fabric, the method including the steps of preparing a solution including bacteria, peptone water and NaCl, feeding the solution including bacteria to a nebulizer, generating an aerosol of said bacterial solution and flowing said solution through a cascade impactor to provide a plurality of bacteria colonies in a plurality of plates present in a plurality of stages of said cascade impactor, characterized in that the concentration of NaCl in the peptone water used to prepare said bacterial solution is in the range of 30 g/L to 150 g/L, preferably in the range of 60 g/L to 120 g/L, and in that the temperature of said cascade impactor is in the range of −15° C. to 15° C., preferably in the range of −4° C. to 15° C., and more preferably in the range 0° C. to 12° C.

As used herein, the term “bacterial filtration efficacy” or “BFE” refers to the efficiency of a face mask, e.g., the fabric forming the face mask, to act as a barrier to bacterial penetration. According to EN 14683, bacterial filtration efficacy must be ≥%95 for type I masks, and ≥%98 for type II/type IIR masks.

According to an aspect of the invention, bacteria are diluted in a solution including peptone water and NaCl, wherein the concentration of NaCl in the peptone water used to prepare the bacterial solution is in the range of 30 g/L to 150 g/L, preferably is in the is in the range of 60 g/L to 150 g/L, more preferably is in the range of 60 g/L to 120 g/L.

According to an aspect of the invention, the temperature of said cascade impactor is in the range of −15° C. to 15° C., preferably in the range of −4° C. to 15° C., more preferably in the range of 0° C. to 12° C. In embodiments, the temperature of the cascade impactor may be in the range of 5° C. to 15° C.

It has been observed that by using a concentration of NaCl between 30 g/L and 150 g/L in the peptone water, and at the same time, using a cascade impactor at a temperature in the range from −15° C. to 15° C., it is possible to obtain particles having MPS in the range from 2.7 μm to 3.3 μm in a reproducible and reliable way, thus allowing a correct evaluation of the amount of the bacteria that are blocked by the fabric, i.e., by the mask. Additionally, it has been observed that only the combined use of the mentioned concentration of NaCl in the peptone water and the impactor cooled at the mentioned temperature allows to obtain the required MPS, i.e., in the range from 2.7 μm to 3.3 μm. In fact, it has been observed that, when a peptone water comprising NaCl in an amount between 30 g/L and 150 g/L is used, without cooling the cascade impactor, the obtained MPS is in the range from 2.4 μm to 2.7 μm, which is in general not acceptable, apart from those cases in which the obtained MPS is exactly 2.7 μm. Therefore, it has been observed that the use of a peptone water comprising NaCl in an amount between 30 g/L and 150 g/L, without cooling the cascade impactor provides for results that are not sufficiently reliable. It was also observed that, when a cooled cascade impactor is used, using the known NaCl concentration in the peptone water, the obtained MPS is in the range from 2.4 μm to 2.6 μm, which is not acceptable.

According to embodiments, the method is a method of testing the bacterial filtering efficiency of a woven fabric.

According to embodiments, the aerosol of bacterial solution is generated inside an aerosol chamber, which is in communication with the cascade impactor.

According to embodiments, the method of the invention is suitable to test BFE with respect to different bacterial. According to embodiments, bacteria may be selected from airborne bacteria (i.e., bacteria present in the atmosphere). According to embodiments, the bacteria may be selected from the group consisting of Mycobacterium tuberculosis, Streptococcus pneumoniae, Legionella pneumophilla, Staphylococcus Aureus, Bacillus Subtilis, and Escherichia Coli. According to preferred embodiments, bacteria are Staphylococcus aureus, preferably Staphylococcus aureus ATCC 6538.

According to embodiments, the method is carried out according to EN 14683:2019+AC:2019 E modified to provide an NaCl concentration of the peptone water and a refrigeration of the cascade impactor as mentioned above. According to embodiments, the method is carried out according to EN 14683:2019+AC:2019 E modified to test bacteria different from Staphylococcus aureus. For example, the method is carried out according to EN 14683:2019+AC:2019 E modified to test bacteria be selected from the group consisting of Mycobacterium tuberculosis, Streptococcus pneumoniae, Legionella pneumophilla, Bacillus Subtilis, and Escherichia Coli, in addition to Staphylococcus Aureus. According to embodiments, the method is carried out according to EN 14683:2019+AC:2019 E modified to provide an NaCl concentration of the peptone water and a refrigeration of the cascade impactor as mentioned above, as well as to use bacteria that are optionally different from Staphylococcus aureus.

In embodiments of the method of the invention, the bacterial solution is delivered to the nebulizer, introduced into an aerosol chamber, and drawn through the impactor under vacuum.

The process for testing bacterial filtration efficacy of a fabric may be carried out, in general, as follows. A first positive control, without fabric samples, is carried out. The petri dishes of the impactor are removed and replaced with fresh plates. The fabric sample is placed between the first stage and the inlet cone of the cascade impactor. After all the samples to be tested have been challenged, a further positive control without fabric is carried out. A negative control is also carried out, without fabric, by passing air, without addition of the bacterial challenge, through the cascade impactor. All the plates obtained are incubated, preferably between 35° C. and 39° C., for a time in the range from 20 to 52 h. The Mean Particle Size and Bacterial filtration Efficacy are then determined for each sample of fabric.

In embodiments, for each sample and control run, the number of colonies on each plate are counted and added up to give the total number of CFU collected by the cascade impactor. The “positive hole” conversion table in accordance with the instructions of the cascade impactor manufacturer is used to correct the count for stages 3 to 6. For example, when the cascade impactor is an Andersen impactor, “Table 1: Positive Hole Conversion Table”, reported in the paper of Ariel A. Andersen, “NEW SAMPLER FOR THE COLLECTION, SIZING, AND ENUMERATION OF VIABLE AIRBORNE PARTICLES”, J Bacteriol. 1958 Nov; 76(5): 471-484, at page 474 is used.

For the two positive control runs, the mean of the two totals is calculated, to obtain the “average converted positive control”, as indicated in the formula below.

From the positive control plates the mean particle size (MPS) of bacterial solution aerosol is calculated.

According to embodiments of the method of the invention, the MPS is determined by counting the colonies of the two positive controls, as above discussed. The counted numbers (converted using the positive hole conversion table where applicable) obtained for each plate are multiplied by the hole size coefficient of the cascade impactor, which is different for each stage (e.g., 7 for stage (plate) 1, 4.7 for stage (plate) 2, 3.3 for stage (plate) 3, 2.1 for stage (plate) 4, 1.1 for stage (plate) 5 and 0.65 for stage (plate) 6). The multiplied numbers of each plate are summed, for each positive control, to obtain the total multiplied converted count of the positive controls. Such total multiplied converted count of the positive controls, obtained for each positive control, is divided by the total count of the respective positive control obtained before the multiplication by the hole size coefficient, to obtain the MPS for each positive control run. The mean value of the two MPS values so obtained is the MPS of the particles of the bacterial solution aerosol.

According to embodiments, the Bacterial Filtration Efficiency is then calculated.

The colonies of each plate obtained from a fabric sample are counted (and converted using the positive hole conversion table where applicable) and summed to obtain a “converted mask value”, as indicated in the formula below. The difference between the average converted positive control count (i.e., the “average converted positive control”, mentioned above) and the total converted count of the fabric, i.e., the “converted mask value” mentioned above, is divided by the “average converted positive control”. The obtained result is multiplied by 100 to obtain the BFE expressed as percentage of the number of colony-forming units present in the bacterial challenge aerosol.

According to embodiments, the bacterial filtration efficiency is calculated by the following formula:

$$Bacterial Filtration Efficiency ( BFE ) = \frac{Average converted positive control - Converted Mask Value}{Average converted positive control} \times 100$$

As above mentioned, the method of the invention is characterized in that the concentration of NaCl in the peptone water used to prepare the bacterial solution is in the range of 30 g/L to 150 g/L and in that the temperature of the cascade impactor is in the range of −15° C. to 15° C.

According to embodiments, the NaCl concentration is in the range of 60 g/L to 150 g/L, preferably in the range of 60 g/L to 120 g/L, and more preferably of 100 g/L.

According to embodiments, the temperature of the cascade impactor is in the range of −4° C. to 15° C., preferably of 0° C. to 12° C.

According to embodiments, the temperature of the cascade impactor may be regulated by incubating the cascade impactor in a refrigerator. For example, the cascade impactor may be incubated in a −80° C. refrigerator for at least 5 minutes, or −20° C. for at least 15 minutes, or +4° C. for at least 1 hour.

According to embodiments, in the testing conditions the nebulizer is capable to generate an aerosol of a saline solution, free from bacteria, having Mean Particle Size of 3.0±0.3 μm. For example, saline solutions may be NaCl or NaF (sodium fluoride) solutions.

Examples of suitable nebulizers are jet, ultrasonic and mesh nebulizers. An exemplary suitable nebulizer is the nebulizer OMRON HEALTHCARE Co., Ltd. (Japan), model: NE-C28P-E. This is a jet nebulizer, which provides for a nebulization rate of 0.05 ml/min, and an aerosol mean particle size of 3 μm, measured using a saline solution, free from bacteria.

According to embodiments, the cascade impactor is a six-stages cascade impactor, preferably an Andersen six-stages cascade impactor. The Andersen six-stage cascade impactor, disclosed in Ariel A. Andersen, “NEW SAMPLER FOR THE COLLECTION, SIZING, AND ENUMERATION OF VIABLE AIRBORNE PARTICLES”, J Bacteriol. 1958 Nov; 76(5): 471-484. Each one of the six stages of the impactor includes a petri dish.

Also object of the invention is a method for bringing into a required size range the Mean Particle Size of particles generated by a nebulizer in a device for testing Bacterial Filtration Efficiency of a fabric, the device comprising a nebulizer for generating an aerosol of a bacterial solution and a cascade impactor including a plurality of stages, said method comprising the steps of: generating an aerosol of a solution including bacteria, peptone water an NaCl; flowing said aerosol through the cascade impactor to provide a plurality of bacteria colonies in a plurality of plates present in a plurality of stages of the cascade impactor, characterized in comprising the steps of: performing at least one positive control run by feeding said aerosol to the cascade impactor; determining the Mean Particle Size of the aerosol particles based on the resulting number of bacterial colonies; comparing the obtained Mean Particle Size with a required range of MPS; modifying the control run conditions by increasing or decreasing the concentration of NaCl in said bacterial solution and/or the temperature of the said cascade impactor; performing at least another positive control run under said modified conditions and repeating said steps until the resulting Mean Particle Size, i.e., a the obtained value of Mean Particle Size, is within the required range. According to embodiments, the required range for the Mean Particle Size is from 2.7 μm to 3.3 μm.

According to embodiments, the method for bringing into a required size range the Mean Particle Size of particles generated by a nebulizer in a device for testing Bacterial Filtration Efficiency of a fabric is carried out according to EN 14683:2019+AC:2019 E. In embodiments, when the method for bringing into a required size range the Mean Particle Size of particles generated by a nebulizer in a device for testing Bacterial Filtration Efficiency of a fabric is carried out according to EN 14683:2019+AC:2019 E, run conditions are optionally modified by increasing or decreasing the concentration of NaCl in the bacterial solution and/or by increasing or decreasing the temperature of the cascade impactor. In embodiments, the method may be carried out according to EN 14683:2019+AC:2019 E modified, optionally, also to test bacteria different from Staphylococcus aureus; for example, using bacteria be selected from the group consisting of Mycobacterium tuberculosis, Streptococcus pneumoniae, Legionella pneumophilla, Bacillus Subtilis, and Escherichia Coli, in addition to Staphylococcus Aureus. According to embodiments, the method is carried out according to EN 14683:2019+AC:2019 E modified to increase or decrease the NaCl concentration in the bacterial solution and/or the temperature of said cascade impactor, and/or to use bacteria that are optionally different from Staphylococcus aureus.

According to embodiments, the bacteria is Staphylococcus aureus, preferably Staphylococcus aureus AATC 6538.

Further object of the present invention is a device for testing Bacterial Filtration Efficiency of a fabric, the device comprising a nebulizer for generating an aerosol of a bacterial solution and a cascade impactor including a plurality of stages, characterized in comprising refrigerating means to refrigerate the cascade impactor.

According to embodiments, the device is a device according to EN 14683:2019+AC:2019 E. In other words, in embodiments, a device according to EN 14683:2019+AC:2019 E may be provided with refrigerating means to refrigerate the cascade impactor. For example, the refrigerating means may be a refrigerating plate or container onto or into which the cascade impactor is placed.

According to embodiments, the refrigerating means are provided at the exit portion of the cascade impactor.

### EXAMPLES

The following examples relate to exemplary embodiments of the present invention and are to be considered as illustrative and non-limiting with respect of the scope of the invention.

### Example 1

### Reagents and Materials

### Tryptic Soy Agar (TSA)

Tryptic soy agar is a solid culture medium. The composition is reported in Table 1, with reference to the liquid solution used to produce the solid medium.

TABLE 1
(formula/litre)

| | |
|---|---|
| Enzymatic Digest of Casein | 15 g |
| Enzymatic Digest of Soybean Meal | 5 g |
| Sodium Chloride | 5 g |
| Agar | 15 g |
| Distilled water | 1000 ml |
| Final pH | 7.3 ± 0.2 at 25° C. |

### Tryptic Soy Broth (TSB)

Tryptic soy broth is a liquid culture medium. The composition is reported in Table 2.

TABLE 2
(formula/litre)

| | |
|---|---|
| Enzymatic Digest of Casein | 17 g |
| Enzymatic Digest of Soybean Meal | 3 g |
| Sodium Chloride | 5 g |
| Dipotassium phosphate | 2.5 g |
| Dextrose | 2.5 g |
| Distilled water | 1000 ml |
| Final pH | 7.3 ± 0.2 at 25° C. |

### Peptone Water—According to Standard Method EN 14683

Peptone water is a liquid medium used for diluting the bacterial culture before the test according to standard method EN 14683. The composition is reported in Table 3.

TABLE 3
(formula/litre)

| | |
|---|---|
| Peptone | 10 g |
| Sodium Chloride (NaCl) | 5 g |
| Distilled water | 1000 ml |
| Final pH | 7.2 ± 0.2 at 25° C. |

Peptone water—According to the Invention
Peptone water according to the invention is a liquid medium used for diluting the bacterial culture before the test according to the invention. The composition of an exemplary embodiment is reported in Table 4.

TABLE 4
(formula/litre)

| | |
|---|---|
| Peptone | 10 g |
| Sodium Chloride (NaCl) | 100 g |
| Distilled water | 1000 ml |
| Final pH | 7.2 ± 0.2 at 25° C. |

### Example 2

### Exemplary Procedure

According to the invention, the cascade impactor is cooled to a temperature in the range of —15° C. to 15° C., for example, by keeping the impactor for at least 5 minutes at —80° C. in a refrigerator.

Additionally, according to the invention, peptone water includes NaCl at a concentration of in the range of 30 g/L to 150 g/L.

### 1) Preparation of Media

a. Liquid media
i. Tryptic Soy Broth (TSB)
ii. Peptone Water

b. Solid medium
i. Tryptic Soy Agar (TSA)

### 2) Bacterial Growth

a. Resuscitation of Staphylococcus aureus bacteria
i. Growth of S. aureus in TSB medium at 37° C. overnight.

b. Inoculation of Staphylococcus aureus bacteria using streak plate method
i. Growth of S. aureus in TSA medium at 37° C. overnight.

### 3) Test Phase

a. Conditioning of test fabric
i. Hanging the test fabric in the climate chamber which provides constant temperature (21 ±5° C.) and relative humidity (85±5%) for at least 4 hours.

b. Inoculation of Staphylococcus aureus bacteria in TSB medium
i. Selecting a single colony of S. aureus bacteria using inoculation loop, adding that colony in TSB medium, and growing bacteria at 37° C. for 4 hours.

c. Dilution of Staphylococcus aureus bacteria
i. Confirmation of adequate growth of bacteria by measuring turbidity of bacterial solution using McFarland densitometer and/or OD600 spectrophotometer.
ii. Diluting bacterial solution with peptone water to obtain 5×10⁵ colony forming unit (cfu).

d. Test operation
i. Calibrating Bacterial Filtration Efficiency (BFE) device by setting vacuum flow rate to 28.3 L/min (1 cubic foot per minute) and running for 2 minutes (1 minute for bacterial challenge and the other one minute for maintaining the airflow without running the nebulizer).
ii. Order of bacterial filtration efficiency tests is as follows (Table 5):

TABLE 5
ORDER OF BACTERIAL FILTRATION EFFICIENCY TEST

| 1 | 2 | 3 | 4 | 5 | 6 | 7 | 8 |
|---|---|---|---|---|---|---|---|
| Positive | Mask | Mask | Mask | Mask | Mask | Positive | Negative |
| control | 1 | 2 | 3 | 4 | 5 | control | control |
| 1 | | | | | | 2 | |

iii. Numbering TSA petri plates from “Control 1-1” to “Control 1-6” with permanent pen, then locating them into Andersen Cascade Impactor (ACI) accordingly.
iv. Pouring bacterial solution into nebulizer chamber.
v. Running the test without any test fabric for 2 minutes to obtain positive control.
vi. Removing TSA petri plates from ACI.
vii. Taking the test fabric (i.e., the test mask) from climate chamber.
viii. Removing ear loop and cutting two sides to open the pleated part of test fabric (i.e., the test mask). The test area shall be minimum 49 cm².
ix. Numbering TSA petri plates from “Mask 1-1” to “Mask 1-6” with permanent pen, then locating them into Andersen Cascade Impactor (ACI) accordingly.
x. Locating test fabric to the top of ACI, and clamping tightly to prevent any leakage.
xi. Running the test with test fabric for 2 minutes (1 minute for bacterial challenge and the other one minute for maintaining the airflow without running the nebulizer) to obtain mask 1 result.
xii. Repeat the same protocol until 5^{th} mask.
xiii. Running the second positive control without any fabric (mask).
xiv. Cleaning the nebulizer from bacterial solution, then running for negative control without mask and bacteria.

e. Growth of Staphylococcus aureus bacteria in incubator
i. Locating all petri plates (8 set×6 plates=48 plates) at incubator at 37° C.±2° C. for between 20 hours and 52 hours.

### 4) Preparation of Report

a. Counting of Staphylococcus aureus bacteria
i. Place TSA petri plate on a black surface, then starting to count by permanent marker or Promega Colony Count app depending on the urgency.
ii. Write down all bacteria number on a paper report.
iii. Transfer all data to Excel, then convert all data except petri 1 and petri 2 according to the positive hole conversion table from the cascade impactor manual. In the present example, an Andersen cascade impactor and the related “Table 1: Positive Hole Conversion Table”, reported in the paper of Ariel A. Andersen, “NEW SAMPLER FOR THE COLLECTION, SIZING, AND ENUMERATION OF VIABLE AIRBORNE PARTICLES”, J Bacteriol. 1958 Nov; 76(5): 471-484, at page 474 were used. In the mentioned table, the Actual bacteria number is represented as “r”, converted bacteria number is represented as “P”. For example, if actual bacteria number is 121, then converted bacteria number is 144.
iv. Numbers in italics represents no conversion, underlines represent summation of converted positive control.

TABLE 6
CONVERSION OF ACTUAL POSITIVE CONTROL
USING “POSITIVE HOLE CONVERSION TABLE”

| | Petri | Petri | Petri | Petri | Petri | Petri | |
|---|---|---|---|---|---|---|---|
| | 1 | 2 | 3 | 4 | 5 | 6 | |
| | (CFU/ | (CFU/ | (CFU/ | (CFU/ | (CFU/ | (CFU/ | Total |
| | plate) | plate) | plate) | plate) | plate) | plate) | CFU |
| Actual | 124 | 210 | 251 | 312 | 283 | 9 | 1189 |
| Positive | | | | | | | |
| Control | | | | | | | |
| 1 | | | | | | | |
| Actual | 134 | 293 | 332 | 366 | 280 | 15 | 1420 |
| Positive | | | | | | | |
| Control | | | | | | | |
| 2 | | | | | | | |
| Con- | 124 | 210 | 395 | 606 | 492 | 9 | 1836 |
| verted | | | | | | | |
| Positive | | | | | | | |
| Control | | | | | | | |
| 1 | | | | | | | |
| Con- | 134 | 293 | 709 | 986 | 482 | 15 | 2619 |
| verted | | | | | | | |
| Positive | | | | | | | |
| Control | | | | | | | |
| 2 | | | | | | | |
| Average | 129 | 251.5 | 552 | 796 | 487 | 12 | 2227.5 |
| Con- | | | | | | | |
| verted | | | | | | | |
| Positive | | | | | | | |
| Control | | | | | | | |

v. Calculation of mean total bacteria number
1. Summation of converted positive control 1.
2. Summation of converted positive control 2.
3. Calculate the average value of two converted positive controls.
4. According to EN14683 Standard, mean value of two converted positive control must be between 1700 and 3000 cfu.

TABLE 7
CONVERSION OF ACTUAL POSITIVE CONTROL
USING “POSITIVE HOLE CONVERSION TABLE”

| | Petri | Petri | Petri | Petri | Petri | Petri | |
|---|---|---|---|---|---|---|---|
| | 1 | 2 | 3 | 4 | 5 | 6 | |
| | (CFU/ | (CFU/ | (CFU/ | (CFU/ | (CFU/ | (CFU/ | Total |
| | plate) | plate) | plate) | plate) | plate) | plate) | CFU |
| Actual | 124 | 210 | 251 | 312 | 283 | 9 | 1189 |
| Positive | | | | | | | |
| Control | | | | | | | |
| 1 | | | | | | | |
| Con- | 124 | 210 | 395 | 606 | 492 | 9 | 1836 |
| verted | | | | | | | |
| Positive | | | | | | | |
| Control | 7 | 4.7 | 3.3 | 2.1 | 1.1 | 0.65 | |
| 1 | | | | | | | |
| Hole | | | | | | | |
| Size | | | | | | | |
| coef- | | | | | | | |
| ficient | | | | | | | |
| Multi- | 868 | 987 | 1303.5 | 1272.6 | 541.2 | 5.9 | 4978.2 |
| plied | | | | | | | |
| con- | | | | | | | |
| verted | | | | | | | |
| positive | | | | | | | |
| control | | | | | | | |
| 1 | | | | | | | |

$$Mean particle size 1 ( MPS 1 ) = \frac{4978.2}{1836} = 2.71$$

TABLE 8
CONVERSION OF ACTUAL POSITIVE CONTROL USING
“POSITIVE HOLE CONVERSION TABLE”

| | Petri | Petri | Petri | Petri | Petri | Petri | |
|---|---|---|---|---|---|---|---|
| | 1 | 2 | 3 | 4 | 5 | 6 | |
| | (CFU/ | (CFU/ | (CFU/ | (CFU/ | (CFU/ | (CFU/ | Total |
| | plate) | plate) | plate) | plate) | plate) | plate) | CFU |
| Actual | 134 | 293 | 332 | 366 | 280 | 15 | 1420 |
| Positive | | | | | | | |
| Control | | | | | | | |
| 2 | | | | | | | |
| Con- | 134 | 293 | 709 | 986 | 482 | 15 | 2619 |
| verted | | | | | | | |
| Positive | | | | | | | |
| Control | 7 | 4.7 | 3.3 | 2.1 | 1.1 | 0.65 | |
| 2 | | | | | | | |
| Hole | | | | | | | |
| Size | | | | | | | |
| coef- | | | | | | | |
| ficient | | | | | | | |
| Multi- | 938 | 1377.1 | 2339.7 | 2070.6 | 530.2 | 9.8 | 7265.4 |
| plied | | | | | | | |
| con- | | | | | | | |
| verted | | | | | | | |
| positive | | | | | | | |
| control | | | | | | | |
| 2 | | | | | | | |

$$Mean particle size 2 ( MPS 2 ) = \frac{7265 , 4}{2619} = 2.77$$

$$Average Mean particle size ( MPS ) = \frac{2.71 + 2.77}{2} = 2.74$$

vi. Calculation of mean particle size
   1. Convert actual bacteria number of two positive controls using the Positive Hole Conversion Table (in the present example, the “Table 1: Positive Hole Conversion Table”, reported in the paper of Ariel A. Andersen, mentioned above, was used).
   2. Multiply converted bacteria numbers from positive control plate 1 to 6 with the respective hole size coefficient, i.e., in this case, 7, 4.7, 3.3, 2.1, 1.1, and 0.65 respectively.
   3. Summation of multiplied numbers for positive control 1
   4. Summation of multiplied numbers for positive control 2
   5. Divide total value of the two multiplied numbers of the two control runs with the total value of the two converted positive controls, to obtain MPS for each positive control.
   6. calculate the Mean value of MPS between the two MPS for positive controls.
   7. According to 14683 Standard, mean particle size must be between 2.7 μm and 3.3 μm.

TABLE 9
CONVERSION OF ACTUAL MASK VALUE
USING “POSITIVE HOLE CONVERSION TABLE”

| | Petri 1 | Petri 2 | Petri 3 | Petri 4 | Petri 5 | Petri 6 | |
|---|---|---|---|---|---|---|---|
| | (CFU/ | (CFU/ | (CFU/ | (CFU/ | (CFU/ | (CFU/ | Total |
| | plate) | plate) | plate) | plate) | plate) | plate) | CFU |
| Actual | 1 | 1 | 15 | 13 | 51 | 15 | 96 |
| Mask | | | | | | | |
| Value | | | | | | | |
| Converted | 1 | 1 | 15 | 13 | 55 | 15 | 100 |
| Mask | | | | | | | |
| Value | | | | | | | |

$$Bacterial Filtration Efficiency ( BFE ) = \frac{Average converted positive control - Converted Mask Value}{Average converted positive control} \times 100$$

$$Bacterial Filtration Efficiency ( BFE ) = \frac{2227.5 - 100}{2227.5} \times = 95.51 \%$$

vii. Calculation of bacterial filtration efficiency
   1. write down all bacteria number on a paper report
   2. transfer all data to Excel, then convert all data except petri 1 and petri 2 according to “Table 1: positive Hole Conversion Table”, as above mentioned
   3. Use converted mask data and average converted positive control for % BFE calculation for each fabric (mask) tested (an example of fabric data for one mask is reported in Table 9, wherein numbers in italics represents no conversion, underlines represent summation of converted fabric values). In order to obtain the final BFE result, 5 mask (i.e., 5 fabric samples) should be tested and averaged.
   4. According to EN 14683 Standard, bacterial filtration efficacy must be ≥% 95 for type I masks, and ≥% 98 for type II/type IIR masks.

### Example 3

### Comparison Between EN 14683 and the Method of the Invention

In the present example, several samples of the same woven fabric were tested.

In the test carried out according to the invention, the same conditions of the test carried out according to EN 14683 were used, except for concentration of NaCl in peptone water and for the temperature of the impactor, that were according to the method of the invention.

### BFE Test With EN 14683

All the solutions and conditions were according to EN 14683. It was found that average mean particle size is 1.81 μm and average BFE result is 78,15%.

TABLE 10
Total and converted cfu values

| | Petri | Petri | Petri | Petri | Petri | Petri | Total | Converted |
|---|---|---|---|---|---|---|---|---|
| | 1 | 2 | 3 | 4 | 5 | 6 | cfu | total (cfu) |
| Positive | 48 | 43 | 262 | 355 | 366 | 254 | 1328 | 2780 |
| control 1 | | | | | | | | |
| Sample 1 | 1 | 6 | 8 | 44 | 198 | 175 | 432 | 565 |
| Sample 2 | 3 | 8 | 9 | 52 | 201 | 183 | 456 | 600 |
| Sample 3 | 2 | 5 | 7 | 33 | 191 | 188 | 426 | 562 |
| Sample 4 | 1 | 2 | 7 | 35 | 177 | 190 | 412 | 539 |
| Sample 5 | 0 | 1 | 5 | 30 | 169 | 193 | 398 | 520 |
| Positive | 31 | 41 | 200 | 338 | 357 | 225 | 1192 | 2318 |
| control 2 | | | | | | | | |

TABLE 11
Average Mean particle size and Converted total bacteria.

| | Positive | Positive | |
|---|---|---|---|
| | control 1 | control 2 | Average |
| Converted total bacteria (cfu) | 2780 | 2318 | 2549 |
| Mean particle size (μm) | 1.84 | 1.76 | 1.81 |

TABLE 12
Bacteria Filtration Efficiency (%)
according to EN 14683 method

| | |
|---|---|
| Sample 1 | 77.83 |
| Sample 2 | 76.46 |
| Sample 3 | 77.95 |
| Sample 4 | 78.85 |
| Sample 5 | 79.60 |
| Average | 78.14 |

### BFE Trial With the Method of the Invention

As above mentioned, the method of the invention allows to maintain the mean particle size in the range between 2.7 μm and 3.3 μm. The method of the invention is different from EN14683 standard, in particular, in the percentage (%) of NaCl in the Peptone water, and in the Andersen cascade temperature.

In the present example:

Peptone Water Preparation: Peptone water according to the invention, having the composition disclosed in Table 4, here above reported was used. In particular, a commercial peptone water product (distributed by Condalab) was used. According to the formulation of such product,1.5 grams of powder contain 0.5 gram of NaCl and 1 gram of peptone. 9.5 grams of NaCl were added into the solution to make a 10 gram/100 ml NaCl solution which has a final concentration of 10% NaCl. Solution was autoclaved at 121° C. for 15 minutes.

Andersen Cascade Impactor Preparation: Six stages Andersen Cascade was cooled, in the present example, on the −80° C. refrigerator for 5 minutes.

TABLE 13
total and converted CFU values

| | Petri | Petri | Petri | Petri | Petri | Petri | Total | Converted |
|---|---|---|---|---|---|---|---|---|
| | 1 | 2 | 3 | 4 | 5 | 6 | cfu | total (cfu) |
| Positive | 120 | 235 | 314 | 272 | 292 | 11 | 1244 | 1961 |
| control 1 | | | | | | | | |
| Sample 1 | 0 | 3 | 10 | 93 | 72 | 0 | 178 | 198 |
| Sample 2 | 10 | 9 | 75 | 149 | 3 | 0 | 246 | 291 |
| Sample 3 | 2 | 1 | 18 | 83 | 41 | 0 | 145 | 157 |
| Sample 4 | 13 | 1 | 26 | 49 | 83 | 0 | 172 | 186 |
| Sample 5 | 1 | 2 | 37 | 71 | 82 | 2 | 195 | 214 |
| Positive | 201 | 211 | 375 | 302 | 156 | 4 | 1249 | 2286 |
| control 2 | | | | | | | | |

TABLE 14
Average Mean particle size and Converted total bacteria.

| | Positive | Positive | |
|---|---|---|---|
| | control 1 | control 2 | Average |
| Converted total bacteria (cfu) | 1961 | 2286 | 2123.5 |
| Mean particle size (μm) | 2.81 | 3.26 | 3.06 |

TABLE 15
Bacteria Filtration Efficiency (%)
according to the method of the invention

| | |
|---|---|
| Sample 1 | 90.68 |
| Sample 2 | 86.30 |
| Sample 3 | 92.61 |
| Sample 4 | 91.24 |
| Sample 5 | 89.92 |
| Average | 90.15 |

It was found that average mean particle size is 3.06 μm and average BFE result is 90.15%.

### CONCLUSION

A 12% BFE result differences between EN 14683 standard and the method of the invention was observed.

TABLE 16
Comparison of result of EN 14683
and the method of the invention.

| | | Method |
|---|---|---|
| | | of the |
| | EN 14683 | invention |
| Sample 1 | 77.83 | 90.68 |
| Sample 2 | 76.46 | 86.30 |
| Sample 3 | 77.95 | 92.61 |
| Sample 4 | 78.85 | 91.24 |
| Sample 5 | 79.60 | 89.92 |
| Average BFE result | 78.14 | 90.15 |
| Mean Particle Size (MPS) | 1.81 | 3.06 |

1. A method of testing the bacterial filtering efficiency of a fabric, the method including the steps of preparing a solution including bacteria, peptone water and NaCl, feeding said solution including bacteria to a nebulizer, generating an aerosol of said bacterial solution and flowing said solution through a cascade impactor to provide a plurality of bacteria colonies in a plurality of plates present in a plurality of stages of said cascade impactor, characterized in that the concentration of NaCl in the peptone water used to prepare said bacterial solution is in the range of 30 g/L to 150 g/L and in that the temperature of said cascade impactor is in the range of −15° C. to 15° C.

2. A method according to claim 1, wherein said method is carried out according to EN 14683:2019+AC:2019 E modified to provide an NaCl concentration of the peptone water and a refrigeration of the cascade impactor as recited in claim 1.

3. A method according to claim 1, wherein said NaCl concentration is in the range of 60 g/L to 150 g/L.

4. A method according to claim 1, wherein the temperature of the cascade impactor is in the range of −4° C. to 15° C.

5. A method according to claim 1, wherein in the testing conditions said nebulizer is capable to generate an aerosol of a saline solution, free from bacteria, having Mean Particle Size in the range of 2.7 to 3.3 μm.

6. A method according to claim 5, wherein said nebulizer is selected from a jet nebulizer, an ultrasonic nebulizer, and a mesh nebulizer.

7. A method for bringing into a required size range the Mean Particle Size of particles generated by a nebulizer in a device for testing Bacterial Filtration Efficiency of a fabric, the device comprising a nebulizer for generating an aerosol of a bacterial solution and a cascade impactor including a plurality of stages, said method comprising the steps of: generating an aerosol of a solution including bacteria, peptone water an NaCl; flowing said aerosol through the cascade impactor to provide a plurality of bacteria colonies in a plurality of plates present in the plurality of stages of said cascade impactor, characterized in comprising the steps of: performing a positive control run by feeding said aerosol to said cascade impactor; determining the Mean Particle Size of the aerosol particles based on the resulting number of bacterial colonies; comparing the obtained Mean Particle Size with a required range of Mean Particle Size; modifying the control run conditions by increasing or decreasing the concentration of NaCl in said bacterial solution and/or the temperature of said cascade impactor; performing at least another positive control run under said modified conditions and repeating said steps until the resulting Mean Particle Size is within the required range.

8. A method according to claim 7, wherein said required range of said Mean Particle Size is from 2.7 μm to 3.3 μm.

9. A method according to claim 7, wherein said method is carried out according to EN 14683:2019+AC:2019 E.

10. A method according to claim 1, wherein said bacteria is selected from the group consisting of Mycobacterium tuberculosis, Streptococcus pneumoniae, Legionella pneumophilla, Staphylococcus aureus, Bacillus subtilis, and Escherichia coli.

11. A method according to claim 1, wherein said fabric sample is placed between the first stage and the inlet cone of said cascade impactor.

12. A device for testing Bacterial Filtration Efficiency of a fabric, the device comprising a nebulizer for generating an aerosol of a bacterial solution and a cascade impactor including a plurality of stages, characterized in comprising refrigerating means to refrigerate said cascade impactor.

13. A device according to claim 12, wherein said device is a device according to EN 14683:2019+AC:2019 E.

14. A device according to claim 12, wherein said refrigerating means are provided at the exit portion of said cascade impactor.

15. A method according to claim 9, wherein the run conditions are modified by increasing or decreasing the concentration of NaCl in said bacterial solution and/or the temperature of said cascade impactor.

16. A method according to claim 7, wherein said bacteria is selected from the group consisting of Mycobacterium tuberculosis, Streptococcus pneumoniae, Legionella pneumophilla, Staphylococcus aureus, Bacillus subtilis, and Escherichia coli.

17. A method according to claim 7, wherein said fabric sample is placed between the first stage and the inlet cone of said cascade impactor.

18. A device according to claim 13, wherein said device is provided with refrigerating means to refrigerate said cascade impactor.

19. A device according to claim 18, wherein said refrigerating means are provided at the exit portion of said cascade impactor.
